(19) United States
(12) Patent Application Publication
(10) Pub. No.: US 2018/0350466 A1
(43) Pub. Date: Dec. 6, 2018

(54) LONGITUDINAL HEALTH PATIENT PROFILE FOR INCIDENTAL FINDINGS

(71) Applicant: KONINKLIJKE PHILIPS N.V., EINDHOVEN (NL)

(72) Inventors: Lucas Oliveira, Melrose, MA (US); Douglas Henrique Teodoro, Säo Paulo (BR); Gabriel Ryan Mankovich, Boston, MA (US); Ranjith Naveen Tellis, Cambridge, MA (US)

(21) Appl. No.: 15/768,842

(22) PCT Filed: Nov. 4, 2016
(86) PCT No.: PCT/IB2016/056654

§ 371 Date: Apr. 17, 2018

Related U.S. Application Data
(60) Provisional application No. 62/251,125, filed on Nov. 5, 2015.

(51) Int. Cl. G16H 50/20; G16H 30/40; G16H 70/20; G06F 17/27

(52) U.S. Cl. CPC G16H 50/20; G06F 17/2705; G16H 70/20; G16H 30/40

(57) ABSTRACT

A system and method perform the steps of retrieving clinical events for a patient; identifying the clinical events relevant to a clinical guideline for an incidental finding, wherein the incidental finding is an imaging observation tangential to the primary goal for performing an imaging exam; parsing out clinical concepts in the clinical events; clustering the clinical concepts according to the clinical guideline for the incidental finding; creating a longitudinal health patient profile by storing clustered clinical concepts for the identified clinical events relevant to the incidental finding clinical guideline; determining whether to define a new imaging finding from a current imaging exam as an incidental finding; and making follow-up recommendations for the defined incidental finding based on the longitudinal health patient profile and relevant patient clinical information.

### BACKGROUND

Radiologists diagnose diseases and provide statuses for diseases after reading a set of images from an imaging exam, and subsequently make follow-up recommendations based on the reading of the imaging exam. Radiology reports include results of a reading of an imaging exam for a patient, and may also include information regarding suggested follow-up recommendations by radiologists. Exemplary follow-up recommendations may include further imaging studies to improve understanding of the clinical problem, or to detect clinical changes in the patient over time. The failure to perform follow-up recommendations may negatively impact patient clinical outcomes.

Radiologists typically must review and make follow up recommendations for a large number of reviewed imaging exams in order to diagnose and treat patients in an effective manner. The designation “radiologist” is used throughout this description to refer to the individual who is reviewing a patient's medical records, but it will be apparent to those of skill in the art that the individual may alternatively be any other appropriate user, such as a doctor, nurse, or other medical professional.

Radiology reports for imaging exams may also include incidental findings, which are image observations in a radiology report that are tangential and not directly related to the original aims for performing an imaging exam, and attentive management of these incidental findings following identification of these incidental findings may lead to early diagnosis and treatment of diseases. However, when incidental findings are recorded in the radiology reports, often follow-up recommendations specific to clinical guidelines for incidental findings may not be provided. Thus, to timely manage incidental findings and provide follow-up recommendations specific to clinical guidelines for the incidental findings, a method is needed for clearly recording, managing, and communicating guideline-specific follow-up recommendations for incidental findings by the radiologist, to improve patient clinical outcomes, minimize patient radiation exposure, and reduce healthcare costs.

### SUMMARY

A method, comprising: retrieving clinical events for a patient; identifying the clinical events relevant to a clinical guideline for an incidental finding, wherein the incidental finding is an imaging observation tangential to the primary goal for performing an imaging exam; parsing out clinical concepts in the clinical events; clustering the clinical concepts according to the clinical guideline for the incidental finding; creating a longitudinal health patient profile by storing clustered clinical concepts for the identified clinical events relevant to the incidental finding clinical guideline; determining whether to define a new imaging finding from a current imaging exam as an incidental finding; and making follow-up recommendations for the defined incidental finding based on the longitudinal health patient profile and relevant patient clinical information.

A system, comprising: a non-transitory computer readable storage medium storing an executable program; and a processor executing the executable program to cause the processor to: retrieve clinical events for a patient; identify the clinical events relevant to a clinical guideline for an incidental finding, wherein the incidental finding is an imaging observation tangential to the primary goal for performing an imaging exam; parse out clinical concepts in the clinical events; cluster the clinical concepts according to the clinical guideline for the incidental finding; create a longitudinal health patient profile by storing clustered clinical concepts for the identified clinical events relevant to the incidental finding clinical guideline; determine whether to define a new imaging finding from a current imaging exam as an incidental finding; and make follow-up recommendations for the defined incidental finding based on the longitudinal health patient profile and relevant patient clinical information.

A non-transitory computer-readable storage medium including a set of instructions executable by a processor, the set of instructions, when executed by the processor, causing the processor to perform operations, comprising: retrieving clinical events for a patient; identifying the clinical events relevant to a clinical guideline for an incidental finding, wherein the incidental finding is an imaging observation tangential to the primary goal for performing an imaging exam; parsing out clinical concepts in the clinical events; clustering the clinical concepts according to the clinical guideline for the incidental finding; creating a longitudinal health patient profile by storing clustered clinical concepts for the identified clinical events relevant to the incidental finding clinical guideline; determining whether to define a new imaging finding from a current imaging exam as an incidental finding; and making follow-up recommendations for the defined incidental finding based on the longitudinal health patient profile and relevant patient clinical information.

### BRIEF DESCRIPTION OF THE DRAWINGS

FIG. 1 shows a schematic drawing of a system according to an exemplary embodiment.

FIG. 2 shows a flow diagram of a method for making follow-up recommendations for an incidental finding, according to a first exemplary embodiment.

FIG. 3 shows a flow diagram of an exemplary method of applying the generated Longitudinal Health Patient Profile (LHPP) to make follow-up recommendations for the incidental finding, from step 208 in FIG. 2.

FIG. 4 shows an in-workflow tool display according to a first exemplary embodiment.

### DETAILED DESCRIPTION

The exemplary embodiments may be further understood with reference to the following description and the appended drawings, wherein like elements are referred to with the same reference numerals. The exemplary embodiments relate to systems and methods for automatically creating and updating a Longitudinal Health Patient Profile (LHPP) to define and manage incidental findings (IF), and provide follow-up recommendations for the defined incidental finding. A radiology report, for example, is a reading of results of an imaging exam for the patient and may include relevant information regarding findings in the image along with follow-up recommendations. A finding on an imaging exam is an imaging observation for a point within an imaging area of interest on an image from a current imaging exam. Incidental findings are image observations in a radiology report that are tangential and not directly related to the original aims for performing an imaging exam. Although exemplary embodiments specifically describe identifying clinical events from radiology reports for creating a LHPP profile, it will be understood by those of skill in the art that the systems and methods of the present disclosure may be used to identify clinical events from any type of study or exam within any of a variety of hospital settings. In addition, although exemplary embodiments specifically describe the management of incidental findings and provision of follow-up recommendations by radiologists, it will be understood by those of skill in the art that the systems and methods of the present disclosure may be used by medical professionals within any of a variety of hospital settings.

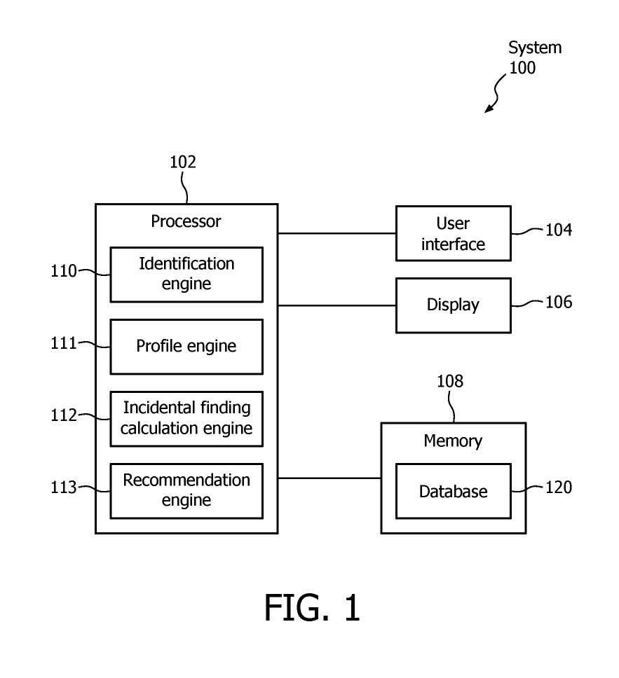

As shown in FIG. 1, a system 100, according to an exemplary embodiment of the present disclosure, creates a Longitudinal Health Patient Profile (LHPP) and manages follow-up recommendations for defined incidental findings (IF) using the LHPP profile, for a patient clinical record. FIG. 1 shows an exemplary system 100 for automatically creating and updating a LHPP profile to manage and provide follow-up recommendations for defined incidental findings (IF), for a patient clinical record. The system 100 comprises a processor 102, a user interface 104, a display 106, and a memory 108. The memory 108 includes a database 120, which stores clinical events located in an electronic medical system, including for example, prior and current imaging exams, drug prescriptions, pathology reports, and radiology reports for a patient. Imaging exams may include exams performed on magnetic resonance imaging (MRI), computed tomography (CT), positron emission chromatography (PET), ultrasound, etc. Those of skill in the art will understand that the method of the present disclosure may be used to create and update a LHPP profile with clinical events from any type of imaging exam or report of an imaging exam. The LHPP profile and the incidental findings for creating and updating the LHPP profile may be viewed in, for example, a display 106, and a radiologist may review and select follow-up recommendations for the incidental findings via a user interface 104.

The processor 102 may be implemented with engines, including, for example, an identification engine 110, a profile engine 111, an incidental finding (IF) calculation engine 112, and a recommendation engine 113. Each of these engines will be described in greater detail below.

Those skilled in the art will understand that the engines 110-113 may be implemented by the processor 102 as, for example, lines of code that are executed by the processor 102, as firmware executed by the processor 102, as a function of the processor 102 being an application specific integrated circuit (ASIC), etc. The identification engine 110 retrieves clinical events from the patient medical record, for example, from the database 120. Exemplary clinical events may include any event stored in an electronic medical system, e.g. electronic medical record (EMR), radiology information system (RIS), etc. The identification engine 110 also identifies relevant clinical events in the patient medical record, relevant to a clinical guideline for an incidental finding, for input into the profile engine 111 to create and update the LHPP profile.

The profile engine 111 creates and updates the LHPP profile. In an exemplary embodiment, the profile engine 111 may initially pre-process the input clinical events by applying a natural language processing parse to parse out and identify clinical concepts within the clinical events, e.g. the clinical concepts of symptoms, diagnoses, and procedures, etc. The profile engine 111 may cluster the identified clinical concepts according to clinical guideline rules for specific incidental findings. For example, the guideline rules for clustering clinical concepts for an incidental lung nodule may be the Fleischner guidelines defining recommendations for the incidental finding of an incidental lung nodule. The profile engine 111 creates the LHPP profile for specific incidental findings by storing clustered clinical concepts for the relevant clinical events, along with clinical guidelines for the specific incidental findings.

The profile engine 111 updates the LHPP profile for specific incidental findings, with additional clustered clinical concepts for the additional relevant clinical events. Returning to the Fleischner guideline example for the incidental lung nodule, in an exemplary embodiment, all clinical concepts associated with smoking history, exposure to asbestos or radon, the family history of lung nodules, and solid or semi-solid mass of the nodule are used to create and update the LHPP profile associated with incidental lung nodules. The incidental finding calculation engine 112 next computes the likelihood a new finding is an incidental finding and determines whether the new finding is an incidental finding, using in-workflow tools or offline processing tools. An exemplary in-workflow tool may be an AIR Ring. In an exemplary embodiment, a radiologist, using an AIR Ring dashboard, identifies and labels a new imaging finding (“new finding”) on the image from an imaging exam. In this exemplary embodiment, the incidental finding calculation engine 112 then determines a confidence level that the new imaging finding is an IF using a multi-factor analysis, including the factors of: the presence of clinical terms stated as reasons for performing an imaging exam, cancer-related clinical terms, and the presence of the new imaging finding in the patient medical history.

The incidental finding calculation engine 112 displays patient clinical information relevant to the new imaging finding for a current imaging exam, with the LHPP profile. In an exemplary embodiment, once a radiologist identifies and labels a new finding, and a LHPP profile is subsequently displayed on display 106 in an in-workflow tool, the radiologist may make a follow-up recommendation for the new imaging finding defined as an incidental finding, based on the LHPP profile, relevant patient clinical information, and clinical guidelines for the incidental finding. In another exemplary embodiment of an in-workflow tool, the recommendation engine 113 may automatically select a follow-up recommendation for a specific incidental finding, based on the LHPP profile and relevant patient clinical information for the defined incidental finding.

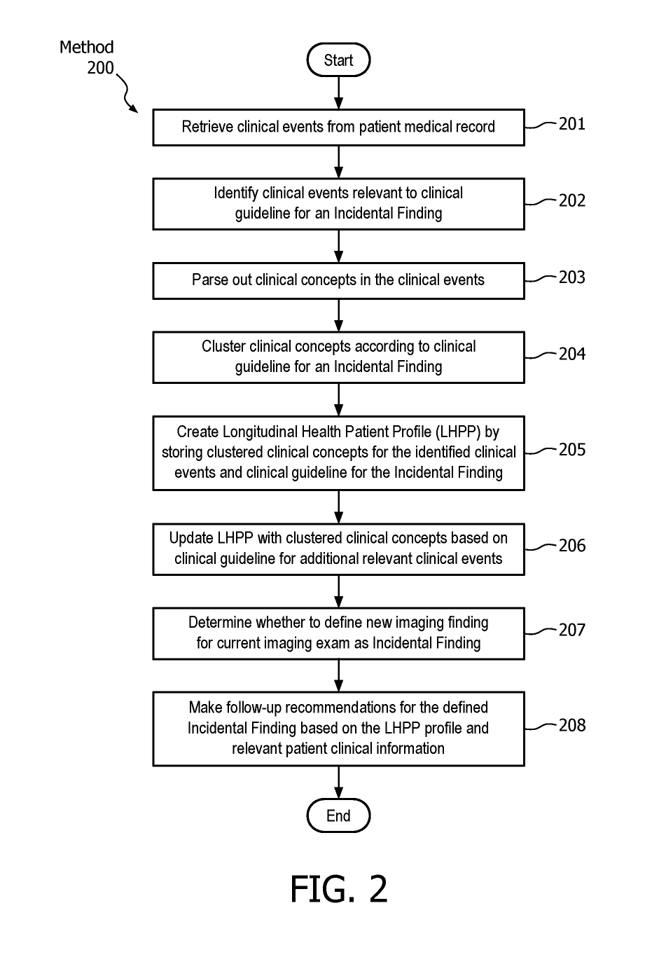

FIG. 2 shows a method 200 for automatically creating and updating a LHPP profile to define and manage incidental findings (IF), and provide follow-up recommendations for the defined incidental finding, for a patient clinical record, using the system 100 above. The method 200 comprises steps for identifying relevant clinical events in the patient medical record, clustering clinical concepts according to clinical guideline rules for incidental findings, creating and updating a Longitudinal Health Patient Profile using the clustered clinical concepts, and determining whether to define a new imaging finding for a current exam as an incidental finding by computing a likelihood that the new imaging finding is an incidental finding.

In step 201, the identification engine 110 retrieves clinical events from the patient medical record. Clinical events may be any event stored in an electronic medical system, e.g. electronic medical record (EMR), radiology information system (RIS), and Laboratory Information System (LIS). Exemplary clinical events may include an updated patient clinical history, new radiology reports, new pathology reports, new pathology results, or the prescription of a drug, etc. In step 202, the identification engine 110 identifies relevant clinical events in the patient medical record, where the identified clinical events are relevant to a clinical guideline for an incidental finding.

In step 203, the profile engine 111 pre-processes the identified clinical events by applying a natural language processing parse, to parse out and identify clinical concepts, e.g. symptoms, diagnoses, and procedures, in the clinical events. In step 204, the profile engine 111 then clusters the identified clinical concepts using a set of clinical guideline rules for a specific incidental finding (IF). An exemplary set of guideline rules for clustering clinical concepts may be the Fleischner guideline defining recommendations for the incidental finding of an incidental lung nodule. Exemplary clustered clinical concepts within the Fleischner guideline for an incidental lung nodule include, for example, smoking history, exposure to asbestos, radon, or uranium, family history of lung nodules, and solid or semi-solid masses of the lung nodule.

In step 205, the profile engine 111 creates the Longitudinal Health Patient Profile (LHPP) profile by storing the clustered clinical concepts for the relevant clinical events, for a specific incidental finding. The LHPP profile is, for example, a context-aware profile storing clinical guidelines and relevant clinical events in the patient medical record, used to aid a healthcare professional in identification and management of incidental findings. For example, a LHPP profile may be created using the Fleischner guideline and relevant patient clinical events for an incidental lung nodule.

In step 206, the profile engine 111 updates the LHPP profile with additional information relevant to a specific incidental finding, including for example, clustered clinical concepts, clinical guidelines, relevant clinical events, patient risk, co-morbidities, and a patient life expectancy, etc. In step 207, the incidental finding calculation engine 112 applies in-workflow tools or offline processing tools to compute the likelihood that the new imaging finding for a current exam is an incidental finding, and determine whether to define the new imaging finding as an incidental finding (IF).

To determine whether to define the new imaging finding as an IF, the incidental finding calculation engine 112 determines the confidence level that a new imaging finding is an IF using a multi-factor analysis. In an exemplary embodiment, to compute the likelihood and confidence level of the new imaging finding as an IF, using the Fleischner guideline, the incidental finding calculation engine 112 considers the factors of: 1) the presence of clinical terms associated with lung disease stated as a reason for performing an exam in a radiology report, e.g. pulmonary nodule, ground glass, or cystic mass; 2) the presence of clinical terms associated with cancer and metastasis, e.g. leukemia, melanoma, and sarcoma; and 3) the presence of any pulmonary lung module in the patient medical record history, e.g. in radiology reports, pathology reports, or other laboratory tests. For example, if the new finding of a pulmonary lung module is recorded for a patient examined for abdominal pain, while the incidental finding calculation engine 112 identifies that previous radiology reports indicate the presence of the cancer-related clinical terms of neck cancer and metastasis in the patient medical record history, the incidental finding calculation engine 112 may determine that the likelihood that the new finding of the lung nodule is an IF is low, and should not be defined as an IF.

In step 208, the incidental finding calculation engine 112 then applies the patient clinical information relevant to the new imaging finding defined as an IF, and the LHPP profile to make follow-up recommendations for an incidental finding. Exemplary follow-up recommendations may include further imaging studies with a different imaging modality. In an exemplary embodiment, a radiologist may confirm the follow-up recommendations generated by incidental finding calculation engine 112, after reviewing the LHPP profile and follow-up recommendations generated by incidental finding calculation engine 112.

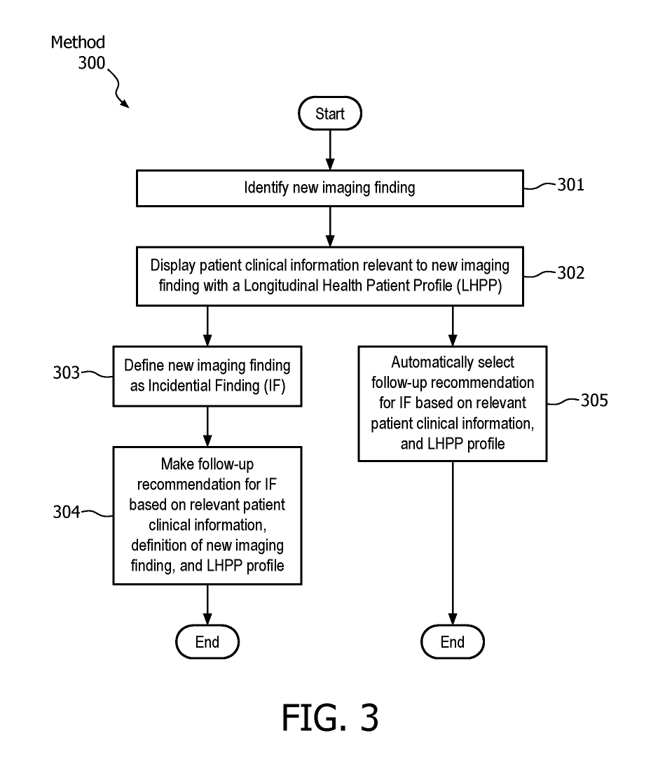

FIG. 3 shows a method 300 for applying the LHPP profile using an in-workflow tool to make follow-up recommendations for an incidental finding, as depicted in step 208 in FIG. 2, depicted in further detail. An exemplary in-workflow tool may be an AIR Ring dashboard. In step 301, using the user interface 104, the radiologist identifies and labels a new imaging finding (“new finding”) on the image from an imaging exam. The new imaging finding is an image observation within a current imaging exam. In an exemplary embodiment, the radiologist identifies a new finding and labels the new finding, e.g. as “left lung nodule” using in-workflow tools, for example, AIR Ring. In step 302, the incidental finding calculation engine 112 displays patient clinical information relevant to the identified new imaging finding with the LHPP profile in an in-workflow tool, displayed on the display 106. Exemplary relevant patient clinical information may include patient risk, co-morbidities for a patient, and patient life expectancy. In an exemplary embodiment, as depicted in steps 302-304, the incidental finding calculation engine 112 displays the LHPP profile in an in-workflow tool on the display 106, for review by a medical professional, e.g. a radiologist. In step 303, the incidental finding calculation engine 112 determines whether to define the new imaging finding as an incidental finding.

In step 304, after the new finding is defined as an incidental finding, the incidental finding calculation engine 112 displays the LHPP profile with relevant patient clinical information and clinical guidelines for the incidental finding, on display 106, to aid the radiologist in making a follow-up recommendation for the selected incidental finding. For example, once a lung nodule is defined as an incidental finding, the incidental finding calculation engine 112 may display on the in-workflow tool, the LHPP profile with a Fleischner clinical guideline and relevant clinical information for the patient, including for example, smoking history, family history of lung cancer, or exposure to asbestos, radon, or uranium, etc. In this exemplary embodiment of an incidental lung nodule, the Fleischner guideline and relevant patient clinical information for an incidental lung nodule are displayed on display 106 to aid the radiologist in making follow-up recommendations for the incidental lung nodule. In an exemplary embodiment, using the user interface 104, the radiologist may click on the LHPP profile displayed on the in-workflow tool to confirm the follow-up recommendations generated by incidental finding calculation engine 112 on the basis of engine 112's definition of a new imaging finding as an incidental finding (IF). For example, after the radiologist identifies and labels a new finding using the in-workflow tool AIR Ring, the AIR Ring dashboard tool may create a dashboard with a LHPP profile and relevant patient clinical information to aid radiologists in confirming the follow-up recommendations generated by incidental finding calculation engine 112, where the recommendations are based on the engine's defined incidental findings.

In an exemplary embodiment, as depicted in step 305, the recommendation engine 113 applies the LHPP profile to automatically select a follow-up recommendation for the selected incidental finding. In an exemplary embodiment, the recommendation engine 113 may apply the Fleischner guidelines on the LHPP profile, along with the relevant clinical information of the lung nodule size to automatically select a follow-up recommendation for the incidental lung nodule, e.g. a follow-up CT scan at 3, 6, and 24 months; dynamic contrast-enhanced CT, PET scans, and a biopsy of the lung nodule.

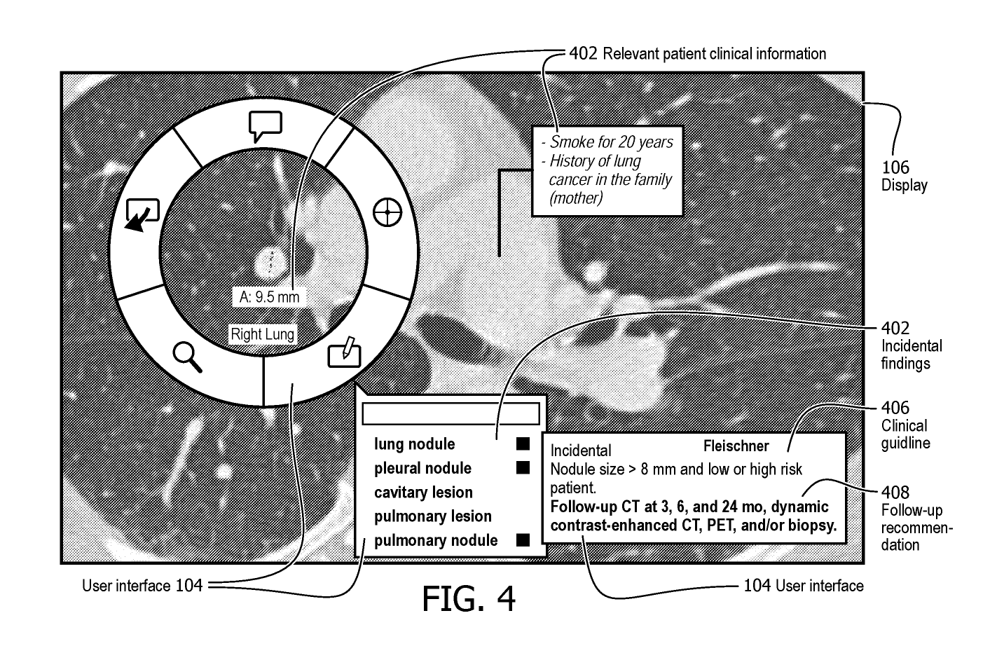

FIG. 4 shows, according to an exemplary embodiment, an in-workflow AIR Ring dashboard tool display 106 presenting relevant clinical information for a patient, along with a LHPP profile with clinical guidelines for an incidental lung nodule, to aid the radiologist in making follow-up recommendations for the incidental lung nodule. In an exemplary embodiment, the radiologist may click on the user interface 104, including the AIR Ring dashboard display of the LHPP profile, to confirm a incidental finding calculation engine 112 definition of the new finding of the lung nodule as an incidental finding, within the section of incidental findings 404. Once the lung nodule is defined as an incidental finding, the incidental finding calculation engine 112 displays, on the display 106, patient clinical information 402 relevant to the lung nodule, e.g. clinical information stating the incidental lung nodule size, patient smoking history, and patient family history of cancer, the LHPP profile, and the clinical guidelines 406 specific to the incidental lung nodule, e.g. the Fleischner guidelines providing follow-up recommendations 408 for the incidental lung nodule. The relevant patient clinical information 402, and LHPP profile with the defined incidental finding, and clinical guidelines 406 with follow-up recommendations 408 are displayed on display 106 to aid the radiologist in making follow-up recommendations for the incidental lung nodule.

Those skilled in the art will understand that the above-described exemplary embodiments may be implemented in any number of manners, including, as a separate software module, as a combination of hardware and software, etc. For example, the identification engine 110, profile engine 111, incidental finding calculation engine 112, and recommendation engine 113 may be programs containing lines of code that, when compiled, may be executed on a processor.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A method, comprising:
retrieving, by a processor comprising an identification engine, clinical events for a patient frown a memory;
identifying, by the identification engine, the clinical events relevant to a clinical guideline for an incidental finding, wherein the incidental finding is an imaging observation tangential to the primary goal for performing an imaging exam;
parsing out, by the processor comprising a findings engine, clinical concepts from the clinical events using natural language processing;
clustering, by the finding engine, the clinical concepts according to the clinical guideline for the incidental finding using natural language processing;
creating, by the findings engine, a longitudinal health patient profile by storing clustered clinical concepts for the identified clinical events relevant to the incidental finding clinical guideline;
determining, by the processor, whether to define a current imaging finding from a current imaging exam as an incidental finding; and
making follow-up recommendations for the defined incidental finding based on the longitudinal health patient profile and relevant patient clinical information.

2. The method of claim 1, further comprising:
updating the longitudinal health patient profile by: inputting additional identified clinical events relevant to the incidental finding;
parsing out clinical concepts from the additional identified clinical events;
clustering the clinical concepts in the additional identified clinical events according to a clinical guideline for the incidental finding; and
updating the longitudinal health patient profile by storing the clustered clinical concepts for the additional identified clinical events.

3. The method of claim 1, wherein the relevant clinical events comprise at least one of:
an updated patient clinical history, new imaging exam reports, a new drug prescription, and new pathology results.

4. The method of claim 1, wherein the clinical guideline lists rules governing potential follow-up recommendations for the incidental finding based on factors comprising at least one of:
patient risk level for the incidental finding, patient risk factors increasing a risk of the incidental finding, size of the incidental finding, physical properties of the incidental finding, and type of imaging exam for the current imaging finding.

5. The method of claim 1, wherein the relevant patient clinical information comprises at least one of:
patient risk level for the incidental finding, co-morbidities in the patient, and patient life expectancy.

6. (canceled)

7. (canceled)

8. The method of claim 1, wherein the determining whether to define a current imaging finding as an incidental finding further comprises computing a likelihood the current imaging finding is the incidental finding, using at least one of:
in-workflow tools; or
offline processing tools.

9. The method of claim 8, wherein the determining whether to define a current imaging finding as an incidental finding further comprises: applying the longitudinal health patient profile with the clinical guideline for the incidental finding and the relevant patient clinical information.

10. (canceled)

11. The method of claim 1, wherein the making follow-up recommendations for the defined incidental finding further comprises:
displaying the longitudinal health patient profile with relevant clinical events;
displaying the relevant patient clinical information;
defining the current imaging finding as the incidental finding;
displaying potential follow-up recommendations listed in the clinical guideline; and
applying the displayed longitudinal health patient profile and the displayed relevant patient clinical information to aid a medical professional in selecting a follow-up recommendation for the incidental finding.

12. The method of claim 1, wherein the making follow-up recommendations for the defined incidental finding further comprises:
applying the displayed longitudinal health patient profile and the relevant patient clinical information to automatically select a follow-up recommendation for the incidental finding.

13. (canceled)

14. A system, comprising:
a non-transitory computer readable storage medium storing an executable program; and
a processor executing the executable program to cause the processor to:
retrieve clinical events for a patient;
identify the clinical events relevant to a clinical guideline for an incidental finding, wherein the incidental finding is an imaging observation tangential to the primary goal for performing an imaging exam;
parse out clinical concepts from the clinical events using natural language processing;
cluster the clinical concepts according to the clinical guideline for the incidental finding using natural language processing;
create a longitudinal health patient profile by storing clustered clinical concepts for the identified clinical events relevant to the incidental finding clinical guideline;
determine whether to define a current imaging finding from a current imaging exam as an incidental finding; and
make follow-up recommendations for the defined incidental finding based on the longitudinal health patient profile and relevant patient clinical information.

15. The system of claim 14, wherein the processor executes the executable program to cause the processor to:
update the longitudinal health patient profile by:
inputting additional identified clinical events relevant to the incidental finding;
parsing out clinical concepts from the additional identified clinical events;
clustering the clinical concepts in the additional identified clinical events according to a clinical guideline for the incidental finding; and
updating the longitudinal health patient profile by storing the clustered clinical concepts for the additional identified clinical events.

16. The system of claim 14, wherein the relevant clinical events comprise at least one of:
an updated patient clinical history, new imaging exam reports, a new drug prescription, and new pathology results.

17. The system of claim 14, wherein the clinical guideline lists rules governing potential follow-up recommendations for the incidental finding based on factors comprising at least one of: patient risk level for the incidental finding, patient risk factors increasing a risk of the incidental finding, size of the incidental finding, physical properties of the incidental finding, and type of imaging exam for the current imaging finding.

18. The system of claim 14, wherein the making follow-up recommendations for the defined incidental finding further comprises:
displaying the longitudinal health patient profile with relevant clinical events;
displaying the relevant patient clinical information;
defining the current imaging finding as the incidental finding;
displaying potential follow-up recommendations listed in the clinical guideline; and
applying the displayed longitudinal health patient profile and the displayed relevant patient clinical information to aid a medical professional in selecting a follow-up recommendation for the incidental finding.

19. The system of claim 14, wherein the making follow-up recommendations for the defined incidental finding further comprises:
applying the displayed longitudinal health patient profile and the relevant patient clinical information to automatically select a follow-up recommendation for the incidental finding.

20. (canceled)
